(19) United States
(12) Patent Application Publication
(10) Pub. No.: US 2024/0083950 A1
(43) Pub. Date: Mar. 14, 2024

(54) SYNTHESIS PROCESS

(71) Applicant: Xellia Pharmaceuticals APS, Copenhagen (DK)

(72) Inventors: Vidar Bjørnstad, Oslo (NO); Karl James Bonney, Oslo (NO)

(21) Appl. No.: 18/260,672

(22) PCT Filed: Jan. 10, 2022
(86) PCT No.: PCT/EP2022/050354

§ 371 Date: Jul. 7, 2023

(30) Foreign Application Priority Data
Jan. 11, 2021 (DK) PA202100027

(51) Int. Cl. C07K 9/00

(52) U.S. Cl. CPC C07K 9/008

(57) ABSTRACT

An optimized method for synthesizing dalbavancin is provided in which an organic antisolvent such as tert-butyl methyl ether (TBME) or dimethoxyethane (DME) is used to precipitate the product of the esterification of A-40926.

### FIELD OF THE INVENTION

The present invention relates to a method for synthesizing dalbavancin.

### BACKGROUND

Dalbavancin is a semisynthetic lipoglycopeptide and exerts its bactericidal effect by disrupting cell wall biosynthesis. It binds to the D-alanyl-D-alanyl residue on growing peptidoglycan chains and prevents transpeptidation from occurring, preventing peptidoglycan elongation and cell wall formation.

Dalbavancin is manufactured by fermentation of a selected Nonomuraea strain to generate the natural glycopeptide complex A-40926. This precursor is then selectively esterified at the carboxyl group of its sugar moiety, its peptidyl carboxyl group is amidated and the ester of the N-acylaminoglucuronic acid carboxyl group is saponified. The outcome is a compound mixture of two closely related structural families—A and B—that can be further subdivided into a total of five subtypes (see table below)

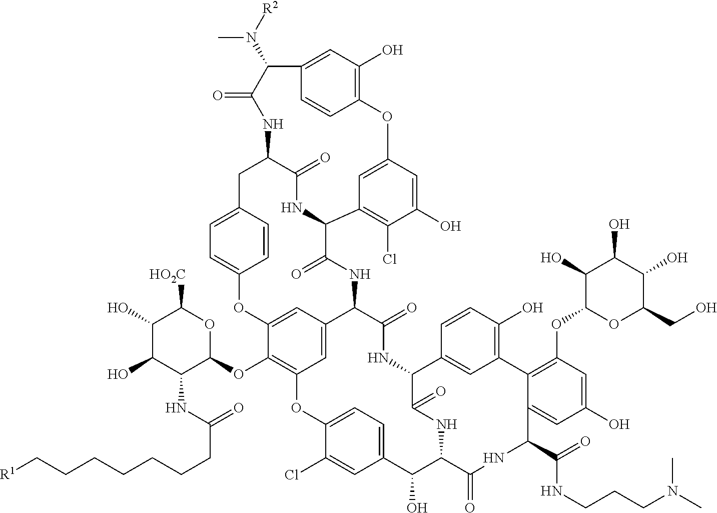

| | Alkyl sidechain of N- | Amino-terminal |
|---|---|---|
| Homolog | acylaminoglucuronic acid (R¹) | substituent (R²) |
| A₀ | CH(CH₃)₂ | H |
| A₁ | CH₂CH₂CH₃ | H |
| B₀ | CH₂CH(CH₃)₂ | H |
| B₁ | CH₂CH₂CH₂CH₃ | H |
| B₂ | CH₂CH(CH₃)₂ | CH₃ |

Various methods exist for converting the precursor A-40926 to dalbavancin, all comprising the steps of esterification, amidation and hydrolysis.

U.S. Pat. No. 6,900,175 describes an esterification step using a solution of sulfuric acid in methanol at 0° C. followed by isolation of the product via pH adjustment with triethylamine to precipitate the zwitterionic form of the product, followed by centrifugation and vacuum-oven drying.

### SUMMARY

The present invention provides an alternative and improved process for synthesizing dalbavancin from the precursor A-40926, the process comprising the steps of:
i) providing a compound of Formula I, which is also denoted A-40926, or a salt thereof

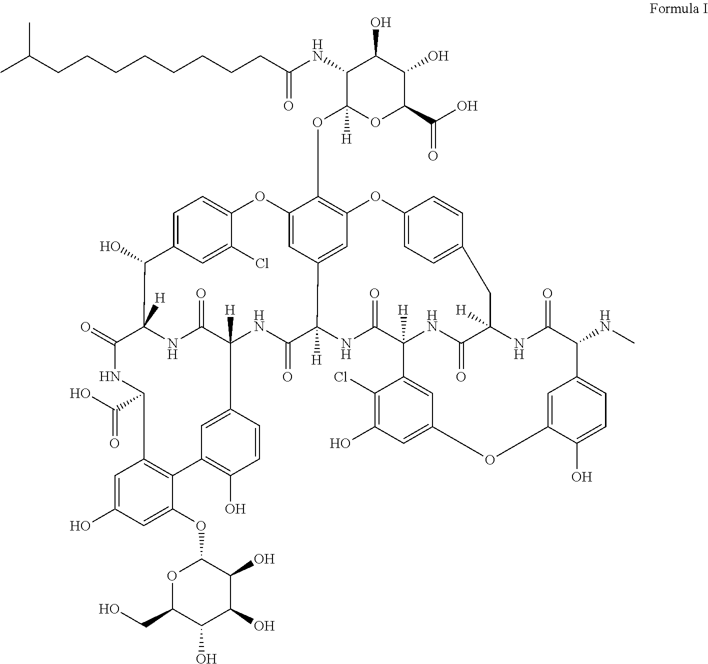 [SMILES: CN[C@H]1C(=O)N[C@@H]2Cc3ccc(cc3)Oc3cc4cc(c3O[C@@H]3O[C@H](C(=O)O)[C@@H](O)[C@H](O)[C@H]3NC(=O)CCCCCCCCC(C)C)Oc3ccc(cc3Cl)[C@@H](O)[C@@H]3NC(=O)[C@H](NC(=O)[C@@H]4NC(=O)[C@@H](NC2=O)c2cc(cc(O)c2Cl)COCc2cc1ccc2O)c1ccc(O)c(c1)-c1c(O[C@H]2O[C@H](CO)[C@@H](O)[C@H](O)[C@@H]2O)cc(O)cc1[C@@H](C(=O)O)NC3=O]

ii) performing an esterification step in order to obtain the compound of Formula II

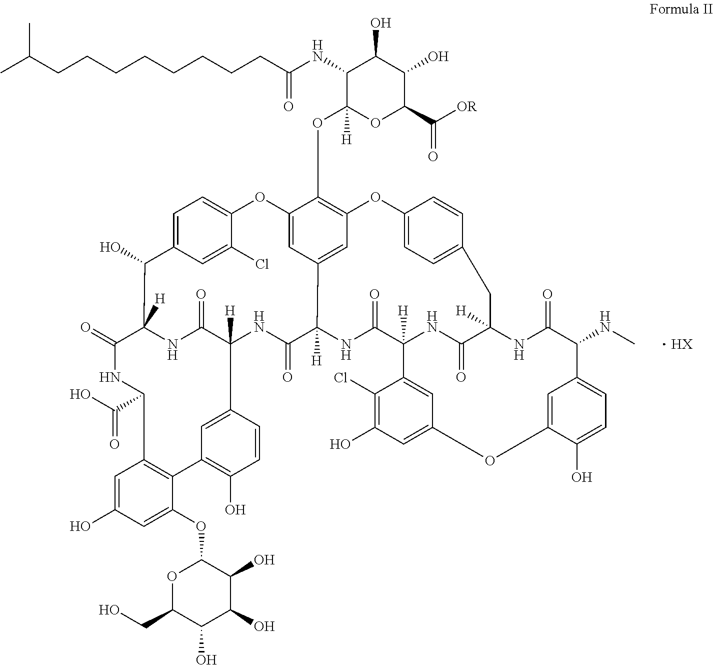

wherein X may be Cl, Br, HSO₄, SO₄, H₂PO₄, HPO₄, PO₄, NO₄, F₃CCO₂, F₃CSO₃, H₃CSO₃ or p-toluenesulfonate and R may be a C₁ to C₆ alkyl group,
iii) adding a suitable amount of tert-butyl methyl ether or dimethoxyethane to form a precipitate,
iv) adding 3-(dimethylamino)-1-propylamine to the precipitate to perform a peptide coupling to obtain a compound of Formula III,

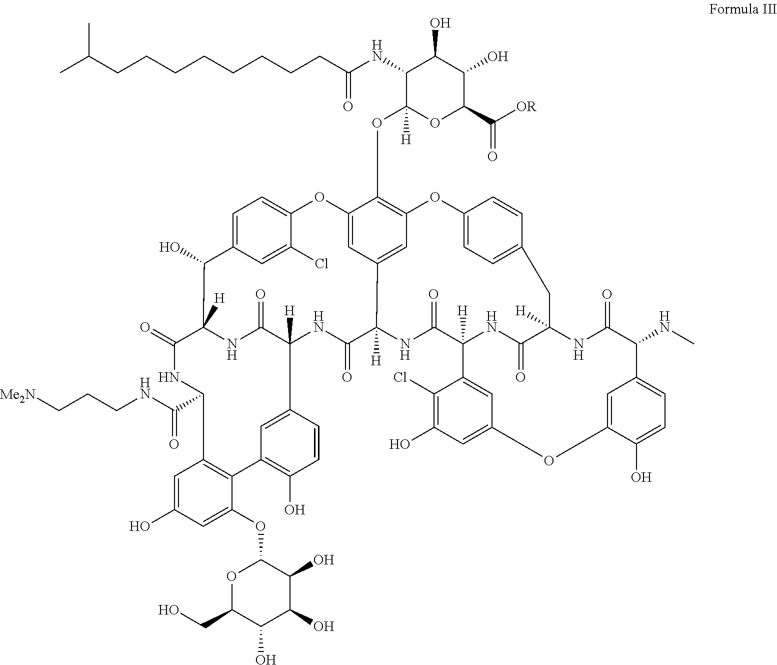

wherein R may be a C₁ to C₆ alkyl group,
v) performing an ester hydrolysis step to obtain dalbavancin or a salt thereof.

### DETAILED DISCLOSURE

In a process as described in the present invention, dalbavancin is prepared from the naturally occurring compound A-40926, which is depicted as Formula I below, through conversion of one of the two carboxyl groups of A-40926 into a (dimethylamino)propyl amide.

In a process according to the invention, dalbavancin is synthesized from the compound in Formula I:

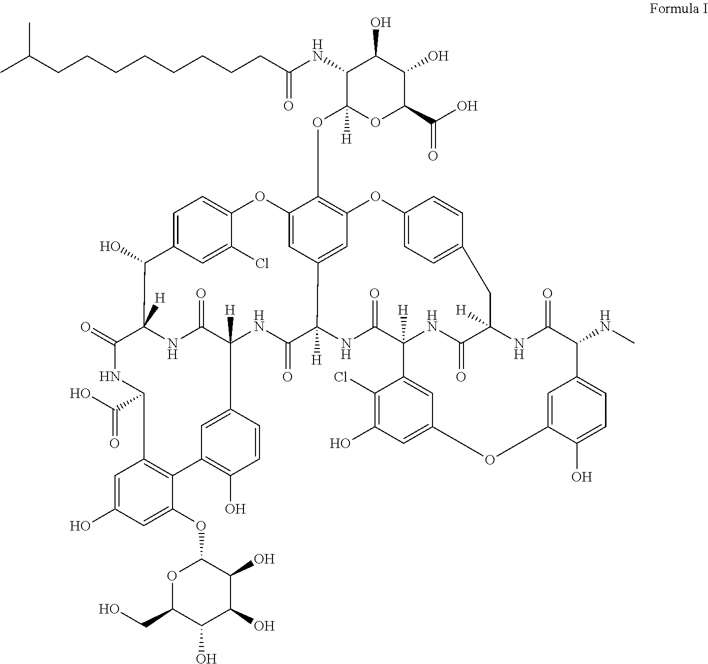 [SMILES: CN[C@H]1C(=O)N[C@@H]2Cc3ccc(cc3)Oc3cc4cc(c3O[C@@H]3O[C@H](C(=O)O)[C@@H](O)[C@H](O)[C@H]3NC(=O)CCCCCCCCC(C)C)Oc3ccc(cc3Cl)[C@@H](O)[C@@H]3NC(=O)[C@H](NC(=O)[C@@H]4NC(=O)[C@@H](NC2=O)c2cc(cc(O)c2Cl)COCc2cc1ccc2O)c1ccc(O)c(c1)-c1c(O[C@H]2O[C@H](CO)[C@@H](O)[C@H](O)[C@@H]2O)cc(O)cc1[C@@H](C(=O)O)NC3=O]

As it appears from Formula I, A-40926 has two carboxylic groups, a peptidyl carboxylic group and a N-acylaminoglucuronic acid group. In order to obtain dalbavancin, the peptidyl carboxylic group is amidated. Accordingly, selective alkyl esterification of the N-acylaminoglucuronic acid group needs to be performed in order to protect the group from amidation and the first step of the synthesis process is an esterification step in order to obtain the compound of Formula II

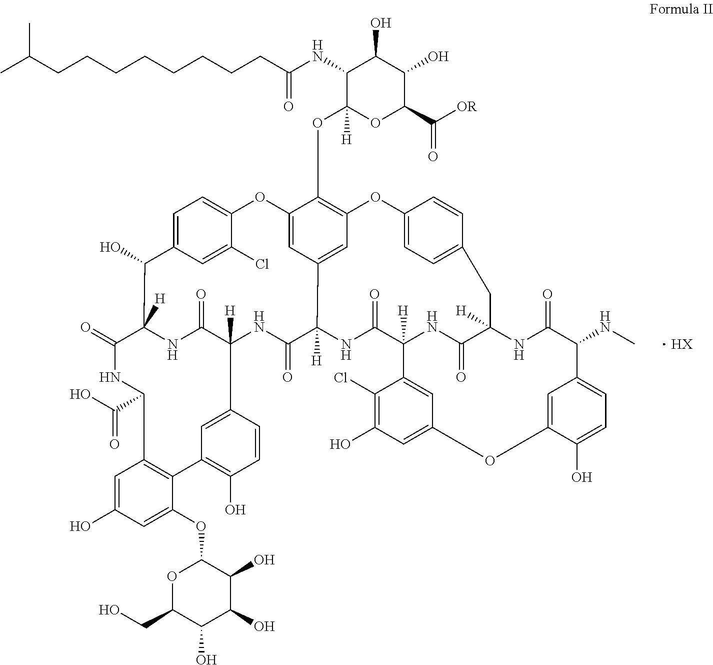

The esterification step is perfomed by adding A-40926 to an alcohol solution comprising an acid, such as for example any of the acids HCl, HBr, H₂SO₄, H₃PO₄, HNO₃, F₃CCO₂H, F₃CSO₃H, H₃CSO₃H or p-toluene sulfonic acid

Accordingly, depending on the acid used, X in Formula II may be Cl, Br, HSO₄, SO₄, H₂PO₄, HPO₄, PO₄, NO₃, F₃CCO₂, F₃CSO₃, H₃CSO₃ or p-toluene sulfonate.

By the term “alcohol solution” is understood a solution comprising an alcohol and wherein not more than 2% water is present. In one embodiment there is not more than 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1% or 0.05%.

In one embodiment the amount of water in the alcohol solution is in the range of 0.05% to 2%.

The alcohol may be methanol, ethanol or a C₃-C₄ alcohol.

R of Formula II may, depending on the alcohol used, be a C₁ to C₆ alkyl group.

By the term “C₁-C₆ alkyl” is meant compounds such as methyl, ethyl, propyl, n-butyl, n-pentyl, n-hexyl, or branched forms thereof.

In one embodiment, the alcohol used is methanol, ethanol or propanol and R is CH₃, CH₃CH₂ or CH₃CH₂CH₂, respectively.

In one embodiment, the alcohol used is methanol and R is CH₃.

As mentioned above, the alcohol solution may further comprise an acid such as, e.g. HCl, HBr, H₂SO₄, H₃PO₄, HNO₃, F₃CCO₂H, F₃CSO₃H, H₃CSO₃ H or p-toluene sulfonic acid.

In one embodiment the acid may be added to the alcohol solution in the form of an anhydrous acid.

However, not all acids are available as anhydrous acids. In one embodiment, the acid may be 15 generated in situ in the alcohol solution by addition of an acyl halide to the alcohol solution.

The term “acyl” is meant to including C₁-C₆ straight or branched alkyl chains. Using an acyl halide to generate an acid in situ as opposed to the addition of an aqueous acid, allows for a faster reaction as the presence of significant quantities of water slows the esterification reaction.

In one embodiment, the halide is chloride, and the acid (HCl) is generated by addition of acyl chloride to the alcohol solution.

In one embodiment, the acyl is acetyl, and the acid is generated by addition of acetyl chloride to the alcohol solution.

In one embodiment of the invention the alcohol is methanol, and acetyl chloride is added for in situ generation of HCl.

In one embodiment, the halide is bromide, and the acid (HBr) is generated by addition of acyl bromide to the alcohol solution.

In one embodiment, the acyl is acetyl, and the acid is generated by addition of acetyl bromide to the alcohol solution.

In one embodiment of the invention the alcohol is methanol, and acetyl bromide is added for in situ generation of HBr.

The esterification step may be performed at a temperature from −20° C. to 10° C., such as, e.g. from −15° C. to 5° C., from −14° C. to 5° C., from −13° C. to 5° C., from −12° C. to 5° C., from −11° C. to 5° C., from −10° C. to 5° C., from −10° C. to 4° C., from −10° C. to 3° C., from −10° C. to 2° C., from −10° C. to −1° C., from −10° C. to 0° C., from −9° C. to 0° C., from −8° C. to 0° C., from −7° C. to 0° C., from −7° C. to −1° C., from −7° C. to −2° C., from −7° C. to −3° C. and from −7° C. to −4° C.

In one embodiment the reaction is performed at a temperature of 5° C., 4° C., 3° C., 2° C., 1° C., 0° C., −1° C., −2° C., −3° C., −4° C., −5° C., −6° C., −7° C., −8° C., −9° C. or −10° C.

The reaction time will depend on the temperature used, and may be from 1 hour to 50 hours, such as, from 1 hour to 45 hours, from 2 hours to 40 hours, from 2 hours to 30 hours, from 2 hours to 24 hours, from 3 hours to 23 hours, from 4 hours to 22 hours and from 5 hours to 21 hours.

The esterification step may be performed using a molar ratio of acetyl chloride:A-40926 of from 30:1 to 5:1, such as, e.g. from 25:1 to 5:1, from 20:1 to 5:1, from 25:1 to 10:1 and from 20 20:1 to 10:1.

In one embodiment the reaction is performed using a molar ratio of acetyl chloride:A-40926 of 20:1, 19:1, 18:1, 17:1, 16:1, 15:1, 14:1, 13:1, 12:1, 11:1 or 10:1.

After the esterification reaction, the resulting intermediate needs to be prepared for the amidation step. In methods known from the art for synthesizing dalbavancin, the intermediate from the esterification step is prepared for the amidation step by precipitation, isolation of the resulting solid and drying in order to remove any unwanted solvents. However, this may lead to an increase in the formation of unwanted impurities, especially of the degradation products mannosyl aglycone (MAG) and the demannosyl derivative (depending on the pH). In addition, methods known in the art involving pH adjustment to precipitate the product may result in a solid that is unsuitable for rapid filtration, and apparently requires thorough drying in a heated vacuum oven to remove all traces of water and alcohol solvent prior to the subsequent amide coupling.

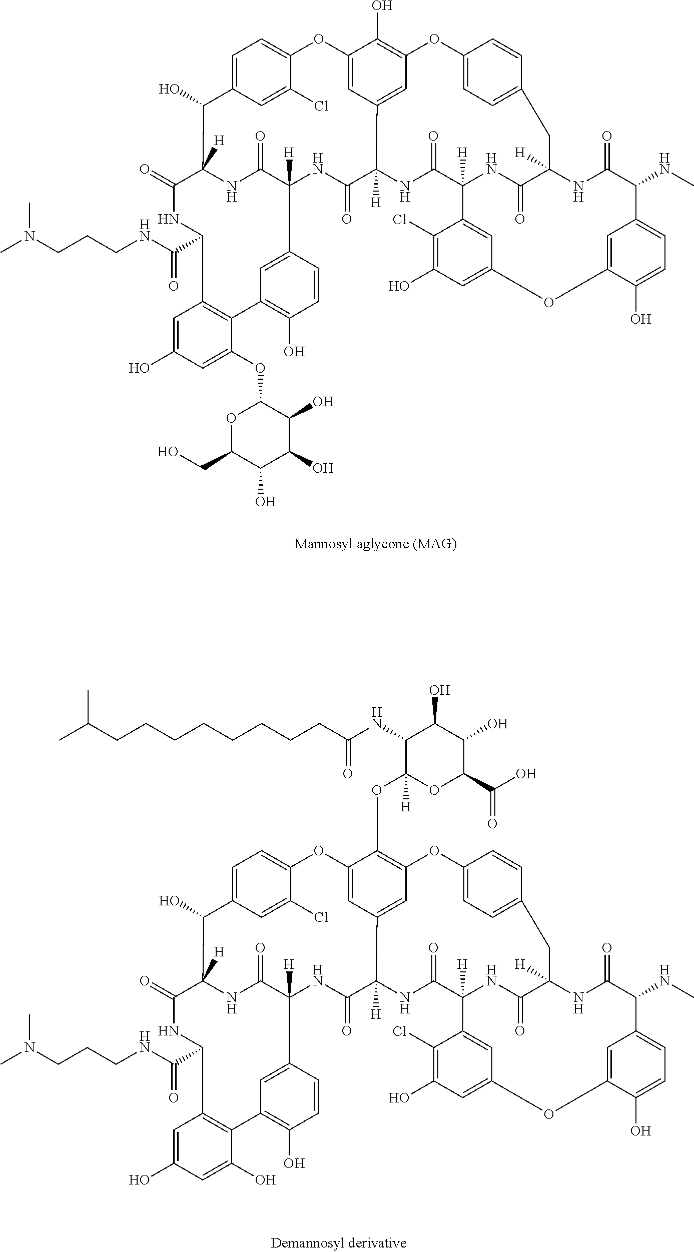

The present inventors have surprisingly found that by using a suitable amount of an organic antisolvent such as tert-butyl methyl ether (TBME) or dimethoxyethane (DME) to precipitate the compound of Formula II, the resulting precipitate can, after a filtration step, be used directly in the following amidation reaction instead of having to further dry the precipitate in an oven, such as a vacuum oven. The inventors have found that by using TBME or DME an easily filterable precipitate is formed, which is suitable for a more facile and time-efficient filtration, and thereby avoiding the need for centrifugation. The inventors have found that the present method generating an easily filterable precipitate is especially suitable for use in an upscale synthesis process and has been used successfully in a scale of 500 g or more of the starting compound A-40926. Other prior known methods may generate a precipitate that may be possible to filter in smaller scale but not possible or practical in larger scale.

20 In one embodiment of the invention the precipitation is performed using TBME. The ratio of alcohol used for esterification to TBME may be from about 1 to 5.0, such as, e.g. from 1 to 4.9, from 1 to 4.8, from 1 to 4.7, from 1 to 4.6, from 1 to 4.5, from 1 to 4.4, from 1 to 4.3, from 1 to 4.2, from 1 to 4.1, from 1 to 4.0, from 1 to 3.9, from 1 to 3.8, from 1 to 3.7, from 1 to 3.6, from 1 to 3.5 and from 1 to 3.4, from 1 to 3.3, from 1 to 3.2, from 1 to 3.1, from 1 to 3.0, from 1 to 2.9, from 1 to 2.8, from 1 to 2.7, from 1 to 2.6, from 1 to 2.5, from 1 to 2.4, from 1 to 2.3, from 1 to 2.2, from 1 to 2.1 and from 1 to 2.0.

The solution comprising the precipitated compound of Formula II may be filtered prior to step iv.

The solution comprising the precipitated compound of Formula II may be filtered by using just gravity.

Accordingly, in on embodiment the filtration step is performed without application of positive or negative pressure to the filter.

The filtration step may be completed by preparing the product of the esterification process for the subsequent amidation reaction by treating it with a flow of gas on the filter, such as, e.g., dry nitrogen gas, a process that is fast and may be completed in approximately an hour and by which the formation of degradation products is avoided, as opposed to e.g., drying in a vacuum oven.

The solution comprising the precipitated compound of Formula II may be filtered using a Nutsche filter. In one embodiment the filtering is performed under gravity, and/or with a high pressure of dry nitrogen gas applied before the filter, and/or with low pressure applied after the Nutsche filter.

After filtering of the solution containing the precipitated compound of Formula II, the precipitate is dissolved in a suitable solvent, such as, e.g. DMSO (dimethyl sulfoxide), DMF (dimethylformide), DMA (dimethylacetamide), THF (tetrahydrofuran), NMP (N-methyl-2-pyrrolidone), or mixtures thereof and a standard amine coupling reagent and 3-(dimethylamino)-1-propylamine were added to the obtained solution in order to obtain a compound of Formula III.

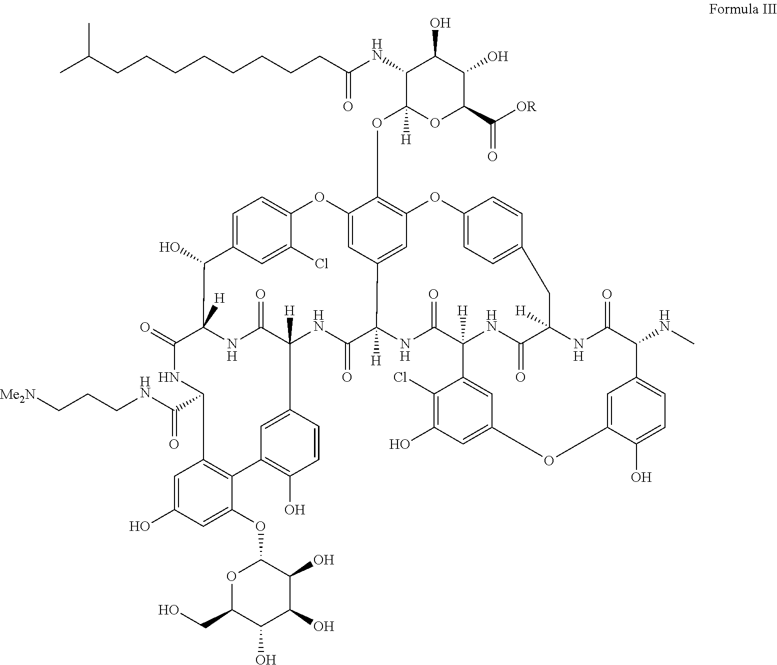

wherein R may be a C₁ to C₆ alkyl group,

Examples of amine coupling reagents that may be used are DCC (dicyclohexylcarboiimide), DIC (diisopropylcarbodiimide), EDC (N-ethyl-N′-(3-dimethylaminopropyl)carbodiimide), HOBt (hydroxybenzotriazole), HOAt (1-hydroxy-7-azabenzotriazole), PyBOP (benzotriazol-1-yl-oxytripyrrolidinophosphonium hexafluorophosphate), HATU (O-(7-Azabenzotriazol-1-yl)-N,N,N′,N′-tetramethyluronium hexafluorophosphate).

After the amine coupling step the compound of Formula II may be obtained by precipitation, filtering and optionally vacuum drying.

In order to obtain dalbavancin, the alkyl group that was added to the N-acylaminoglucuronic acid carboxyl group in the esterification step needs to be removed. This is performed by an ester hydrolysis step.

The dalbavancin obtained by the synthesis process described herein may be further purified in order to remove remaining fermentation related impurities and/or synthesis and process related impurities.

All numbers in the specification and claims are modified by the term “about”. This means that each number includes minor variations as defined ±10% of the numerical value or range in question.

### EXAMPLE 1

(a) A40926 (645 g) was added portion-wise to a solution of methanolic HCl (0.55 M, 10.0 L) at 0° C. under a dry nitrogen atmosphere, at such a rate to ensure the temperature did not exceed 4° C. The temperature was then adjusted to 4° C. and the reaction progress was monitored at intervals by HPLC. When the reaction was judged by HPLC to be complete, TBME (32.0 L) was added over a period of approximately 1 h to precipitate the product. The solid material was filtered using a Nutsche filter and was washed with additional TBME (3×5 L). The filter cake was dried using a flow of dry nitrogen gas, until the solid material had a powder-like consistency. The semi-dried material was used directly in the subsequent reaction.
(b) The semi-dried material (1 equiv.) was dissolved in DMF (75 mM), cooled to 0° C. and triethylamine (2.0 equiv.) and HATU (1.0 equiv.) were added. After 10 min 3-(dimethylamino)-1-propylamine (1.0 equiv.) was added and the mixture was stirred for an additional 30 min. Ethyl acetate was added to precipitate the product, which was filtered and washed with additional ethyl acetate. The solid material was dried in a vacuum oven at 30° C. and used in the subsequent reaction without further purification.
(c) A slurry of the coupled product (667 g) in water (12.5 L) was cooled to 2° C. and aqueous sodium hydroxide (2 M, 1.4 L) was added over 5 min, maintaining the temperature below 4° C. The temperature was then adjusted to 6° C. and the reaction mixture was stirred at this temperature for 3 h.

1. A process for synthesis of dalbavancin comprising the steps of
i) providing a compound of Formula I or a salt thereof 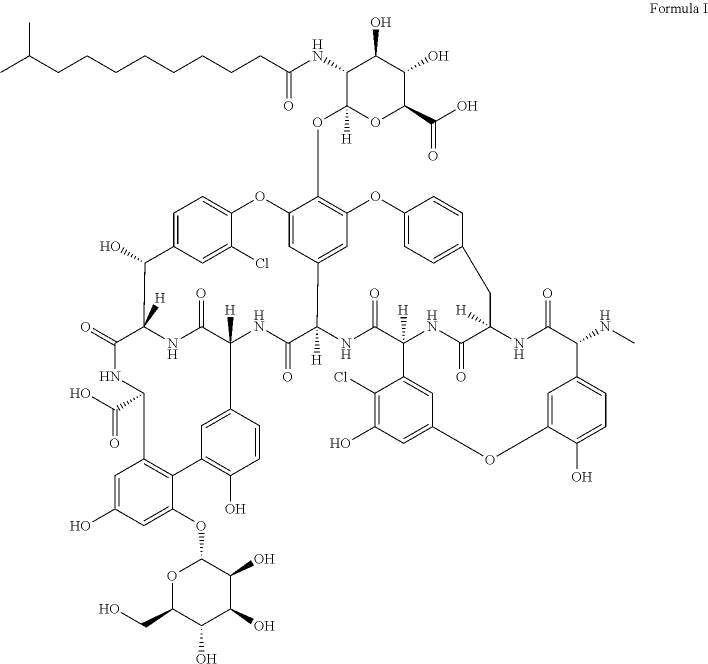 [SMILES: CN[C@H]1C(=O)N[C@@H]2Cc3ccc(cc3)Oc3cc4cc(c3O[C@@H]3O[C@H](C(=O)O)[C@@H](O)[C@H](O)[C@H]3NC(=O)CCCCCCCCC(C)C)Oc3ccc(cc3Cl)[C@@H](O)[C@@H]3NC(=O)[C@H](NC(=O)[C@@H]4NC(=O)[C@@H](NC2=O)c2cc(cc(O)c2Cl)COCc2cc1ccc2O)c1ccc(O)c(c1)-c1c(O[C@H]2O[C@H](CO)[C@@H](O)[C@H](O)[C@@H]2O)cc(O)cc1[C@@H](C(=O)O)NC3=O]
ii) performing an esterification step by adding an alcohol solution comprising an acid to the compound of Formula I in order to obtain the compound of Formula II 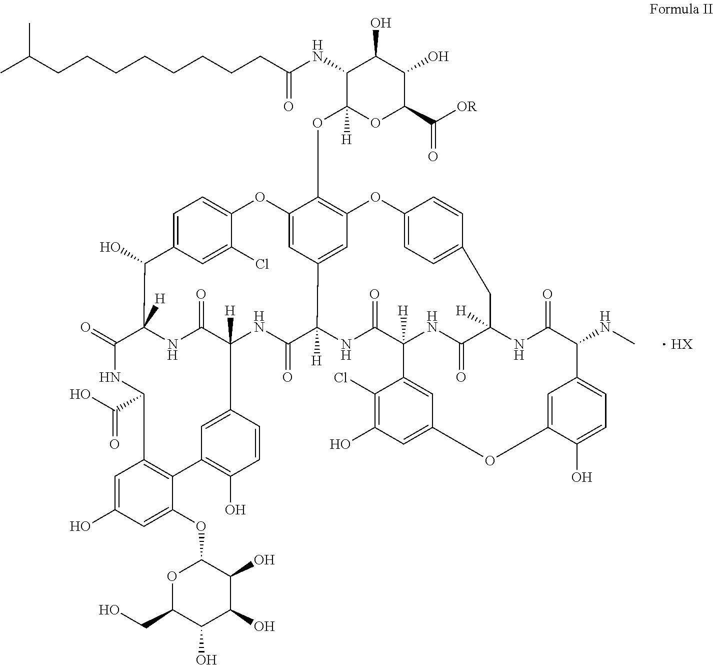
wherein X is Cl, Br, HSO₄, SO₄, H₂PO₄, HPO₄, PO₄, NO₃, F₃CCO₂, F₃CSO₃, H₃CSO₃ or p-toluene sulfonate, and R is a C₁ to C₆ alkyl group,
iii) adding an a amount of tert-butyl methyl ether or dimethoxyethane to form a precipitate
iv) adding 3-(dimethylamino)-1-propylamine to the precipitate to obtain a compound of Formula III 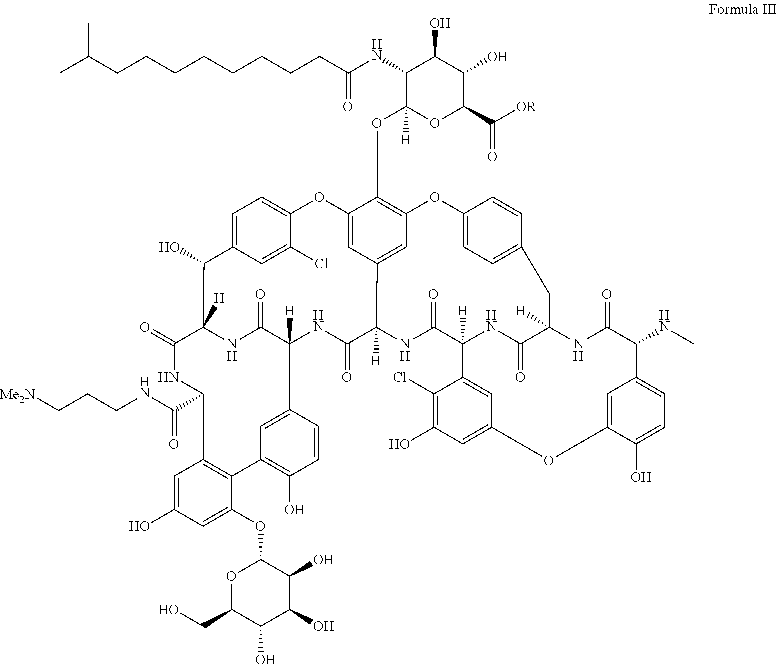
wherein R is a C₁ to C₆ alkyl group, and
v) performing an ester hydrolysis step to obtain dalbavancin or a salt thereof.

2. The process according to claim 1, wherein the precipitation step iii) is performed with tert-butyl methyl ether.

3. The process according to claim 3, wherein the acid in step ii) is an anhydrous acid

4. The process according to claim 1, wherein the acid in step ii) is generated directly in the alcohol solution by addition of an acyl halide to the alcohol solution.

5. The process according to claim 4, wherein the acid is generated by addition of acyl chloride to the alcohol solution.

6. The process according to claim 5, wherein the acid is generated by addition of acetyl chloride to the alcohol solution.

7. The process according to claim 1, wherein X is Cl.

8. The process according to claim 1, wherein X is Br.

9. The process according to claim 4, wherein the acid is generated by addition of acyl bromide to the alcohol solution.

10. The process according to claim 9, wherein the acid is generated by addition of acetyl bromide to the alcohol solution.

11. The process according to claim 1, wherein the alcohol solution in step ii) is a methanol solution.

12. A process according to claim 1, wherein R is a methyl group.
